(19) United States
(12) Patent Application Publication
(10) Pub. No.: US 2020/0282167 A1
(43) Pub. Date: Sep. 10, 2020

(54) NASAL CANNULA FOR A PORTABLE OXYGEN CONCENTRATOR

(71) Applicant: 3B Medical, Inc., Winter Haven, FL (US)

(72) Inventors: Cody James Aardema, White Bear Lake, MN (US); Perry Karl Jagger, Hudson, WI (US)

(21) Appl. No.: 16/793,393

(22) Filed: Feb. 18, 2020

Related U.S. Application Data
(60) Provisional application No. 62/815,559, filed on Mar. 8, 2019.

(51) Int. Cl. A61M 16/06; A61M 16/08

(52) U.S. Cl. CPC A61M 16/0672; A61M 2202/0208; A61M 16/0816

(57) ABSTRACT

A nasal cannula for an oxygen delivery system according to an exemplary aspect of the present disclosure includes, among other things, a cannula having an end configured to attach to an oxygen source and a face piece configured to deliver a fluid from the oxygen source to a user. A magnetic coupling is arranged along the cannula.

### PRIORITY CLAIM

This application claims the benefit of U.S. Provisional Application No. 62/815,559, filed on Mar. 8, 2019.

### TECHNICAL FIELD

This disclosure relates to a nasal cannula for supplying oxygen to a user.

### BACKGROUND

In the medical field, oxygen may be supplied to patients to treat a variety of conditions such as heart failure, Chronic Obstructive Pulmonary Disease (COPD), or any weakened lung or heart state. Portable oxygen concentrators (POCs) are one known device used in the medical field to supply supplemental oxygen to a patient. POCs take in ambient air, filter it, and deliver a relatively high purity flow of oxygen to the patient. At times, supplemental oxygen is used for purposes outside of the medical field, such as for recreational purposes. Supplemental oxygen may be used to shorten recovery time for exhausted athletes, or may be used at high altitudes to make breathing easier during skiing, mountain biking, or other sporting activities.

### BRIEF DESCRIPTION OF THE DRAWINGS

FIG. 1 schematically illustrates an example oxygen delivery system.

FIG. 2 illustrates a portion of the example oxygen delivery system.

FIG. 3 illustrates an example magnetic coupling in a detached state.

FIG. 4 illustrates the example magnetic coupling of FIG. 3 in an attached state.

FIG. 5 schematically illustrates another example oxygen delivery system.

FIG. 6 illustrates, somewhat schematically, yet another example oxygen delivery system.

### SUMMARY

A nasal cannula for an oxygen delivery system according to an exemplary aspect of the present disclosure includes, among other things, a cannula having an end configured to attach to an oxygen source and a face piece configured to deliver a fluid from the oxygen source to a user. A magnetic coupling is arranged along the cannula.

In a further embodiment, the magnetic coupling comprises a first magnet and a second magnet.

In a further embodiment, the first and second magnets have a ring shape.

In a further embodiment, a protrusion is arranged in a center of the first magnet, and a cavity is arranged in a second center of the second magnet, the protrusion configured to engage with the cavity.

In a further embodiment, a seal extends about at least one of the first and second magnets.

In a further embodiment, the first magnet is arranged at the end of the cannula and is configured to engage with the second magnet arranged on the oxygen source.

In a further embodiment, the first and second magnets are arranged along the cannula between the end and the face piece.

In a further embodiment, the fluid is configured to travel through the magnetic coupling.

In a further embodiment, the magnetic coupling comprises a mounting piece having at least one hole configured to mount the magnetic coupling to the oxygen source.

In a further embodiment, the at least one hole is a threaded hole.

In a further embodiment, the magnetic coupling comprises an alignment guide.

In a further embodiment, the oxygen source is a portable oxygen concentrator.

In a further embodiment, the user is an athlete.

A portable oxygen concentrator system according to an exemplary aspect of the present disclosure includes, among other things, a portable oxygen concentrator having a filter arranged within a housing, the filter configured to draw in ambient air, remove other gases, and deliver concentrated oxygen through an outlet on the housing. A first magnet is at the outlet. A cannula having an end with a second magnet is configured to attach to the first magnet.

In a further embodiment, the cannula has a face piece at a second end opposite the end, the face piece configured to deliver the concentrated oxygen to a user.

In a further embodiment, the first and second magnets have a ring shape.

In a further embodiment, the first magnet extends about the outlet.

In a further embodiment, a seal is arranged between the first and second magnets.

In a further embodiment, the cannula is configured to deliver the concentrated oxygen to a user, and the user is an athlete.

In a further embodiment, the portable oxygen concentrator is configured to deliver the concentrated oxygen at a purity of less than 86%.

### DETAILED DESCRIPTION

This disclosure relates to a nasal cannula for supplying oxygen to a user, such as from a portable oxygen concentrator (POC).

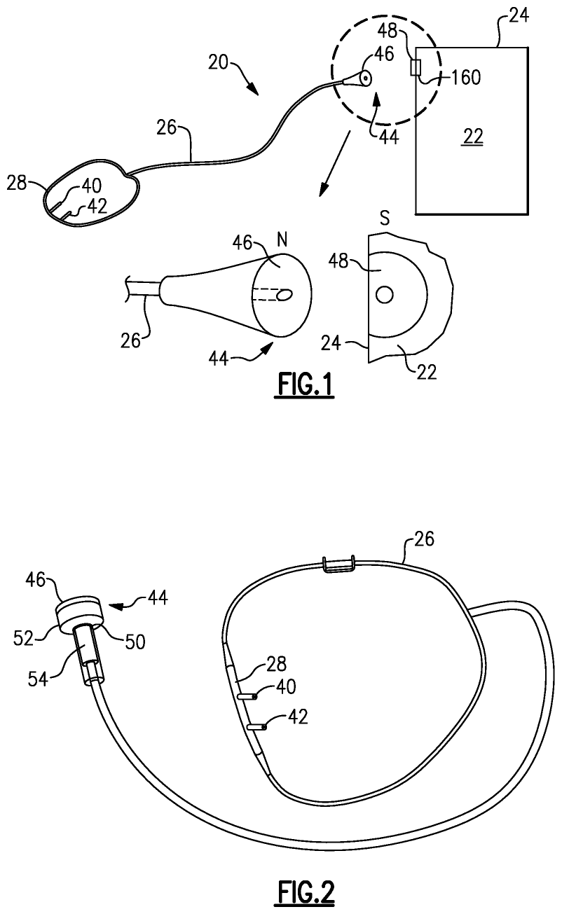

FIG. 1 illustrates an example oxygen delivery system 20, and includes a breakout showing the detail of a magnetic coupling. The system 20 includes a nasal cannula 26 connected to an oxygen source, such as a POC 22. The nasal cannula 26 is configured to deliver oxygen from the POC 22 to a user. Although a POC 22 is shown in FIG. 1, the oxygen source may be any type of oxygen delivery system, such as an oxygen tank, or another breathing aid, such as a continuous positive airway pressure (CPAP) machine.

The POC 22 includes an air compressor, one or more filters, and a battery. In an embodiment, the filter is a molecular sieve which separates (i.e., adsorbs) nitrogen from the ambient air. The POC 22 may use pressure swing adsorption (PSA), vacuum swing adsorption (VSA), or pressure vacuum swing adsorption (PVSA) technology. The POC 22 may further include a storage chamber, or reservoir. The battery may be rechargeable.

The POC 22 delivers oxygen via the cannula 26 to an interface, which in this example is mask or face piece 28, which delivers the oxygen to a user. The POC 22 may be a pulse delivery device or a continuous flow device. A continuous flow POC provides a continuous flow of oxygen to the patient. A pulse delivery POC only provides oxygen when the patient is inhaling. Thus, with pulse delivery, there is a reduced load on the POC compared to a continuous flow device.

Ambient air contains about 21% oxygen and about 79% nitrogen and other gases. The POC 22 compresses the ambient air and filters the nitrogen out of the air, leaving oxygen as the primary gas in the product delivered to the user via the face piece 28. The nitrogen is released back to the ambient environment and/or held in the filters. In a typical medical grade POC, the gas delivered to a patient is around 90-95% oxygen. In other embodiments, such as in POCs for recreational use, a lower oxygen purity is delivered to the patient. The POC 22 may include flow control buttons and indicators for breath detection or alerts, and sometimes includes the ability to toggle between a continuous flow and a pulse flow.

It should be understood that the POC 22 includes a control unit programmed with executable instructions for interfacing with and operating the various components of the POC 22. The control unit is further programmed to provide the other functionality discussed above, among other features. It should be understood that the control unit could be part of an overall control module. The control unit includes a processing unit and non-transitory memory for executing the various control strategies and modes of the POC system.

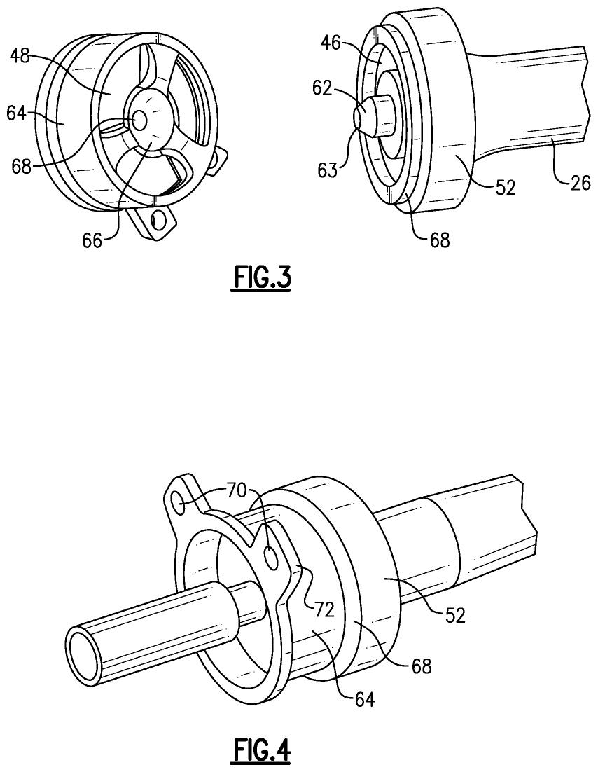

In this example, the cannula 26 wraps around a user's head and has a face piece 28 that rests at the base of the nose for delivery of oxygen. In this example, the face piece 28 includes first and second nostril protrusions 40, 42 (FIGS. 1 and 2) for delivering oxygen to the user's nose. In some embodiments, the cannula 26 is made entirely of silicone, or other soft elastomer. The cannula 26 and face piece 28 may be provided in different sizes to accommodate different users. Although a nasal cannula is shown, other interfaces, such as other facial masks, fall within the scope of this disclosure.

Opposite the face piece 28, the cannula 26 has a magnetic coupling 44 which connects the cannula 26 to the POC 22. In the example of FIGS. 1 and 2, a first magnet 46 is arranged at an end of the cannula 26 and a second magnet 48 is arranged on the POC 22. The first and second magnets 46, 48 have opposite poles such that they attract one another. The first and second magnets 46, 48 are ring shaped to permit the flow of oxygen through the magnetic coupling 44 when the POC 22 is in use. Known POC devices have a threaded attachment for a cannula. The disclosed system 20 has the second magnet 48 secured directly to the POC 22 for attaching the cannula 26. The second magnet 48 may be secured to the POC 22, such as with an adhesive, or may be integrated into a housing 24 of the POC 22 such as by overmolding.

In some examples, one of the magnetic portions 46, 48 is a ferromagnetic material, such as steel, while the other of the magnetic portions 46, 48 is another type of magnetic material. This may provide a less expensive alternative to both magnetic portions 46, 48 being magnetic materials. An alignment guide 160 may be provided on the POC 22 or the cannula 26 to ensure the magnets 46, 48 are aligned. The attraction between the magnets 46, 48 must be strong enough to form a seal at the magnetic coupling 44. This seal permits oxygen to flow through the cannula 26, passing through the magnets 46, 48 before reaching the cannula 26.

As shown in FIG. 2, the first magnet 46 is secured at the end 50 of the cannula 26. In this example, the magnet 46 is attached to a coupling piece 52 having a stem 54. The stem 54 extends into the cannula 26, and is secured within the cannula 26 via a friction fit (press fit). In some examples, the magnet 46 and coupling piece 58 are a single integrated structure. The magnets 46, 48 may be secured to the cannula 26 and/or POC 22 via a friction fit or an adhesive, for example. In other examples, the magnets 46, 48 are over-molded in plastic. This may ensure all materials in the system 20 are medical grade materials.

The magnetic coupling 44 permits a user wearing the face piece 28 to detach and reattach to the POC 22 quickly and easily. The magnetic coupling 44 may also detach when the cannula 26 snags on something, preventing any discomfort to the user from a sudden jerk on the face piece 28.

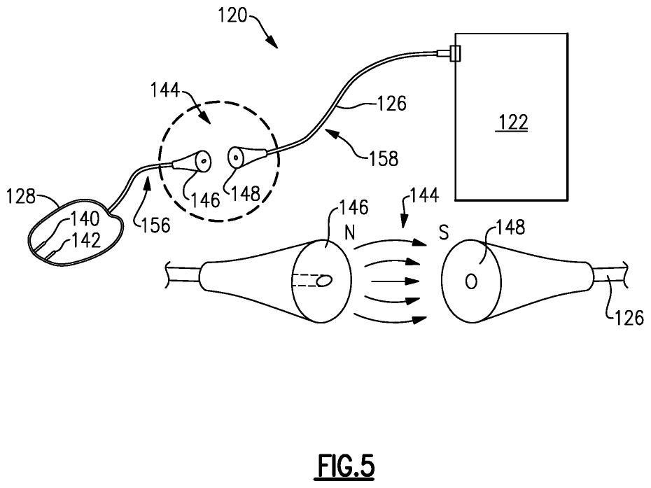

FIGS. 3 and 4 illustrate additional details of an example magnetic coupling. As shown in FIG. 3, the coupling piece 52 may include a protrusion 62 extending outward from a center of the first magnet 46. The protrusion 62 has a through hole 63 for permitting the flow of fluid. The second magnet 48 is secured in place with a mounting piece 64. The mounting piece 64 has a central cavity 66 configured to receive the protrusion 62. In other words, a plane is formed where the magnets 46, 48 attach, and the protrusion 62 extends beyond the plane. A through hole 65 is centered in the cavity 64. The protrusion 62 and cavity 66 help ensure the first and second magnets 46, 48 are properly aligned when attached to one another. This alignment also ensures the holes 63, 65 are aligned to permit fluid to flow through the magnetic coupling 44 from the POC 22 to the face piece 28. Although the protrusion 62 is shown near the first magnet 46 while the cavity 66 is shown near the second magnet 48, these could be switched so that a cavity is near the first magnet 46 while a protrusion is near the second magnet 48. Other locating features may also be utilized in place of a cavity and protrusion.

In this example, the magnets 46, 48 do not contact one another when the magnetic coupling 44 is attached. The magnets 46, 48 may be spaced by at least one of the coupling piece 52 and the mounting piece 64. In the illustrated example, the mounting piece 64 includes three projections that connect the cavity 66 to an outer circumference. The thickness of these projections defines the gap between the magnets 46, 48, in some examples. The magnets 46, 48 and the gap between the magnets is selected such that the attraction between the magnets 46, 48 is strong enough to form a seal at the magnetic coupling 44.

In some examples, a seal 68 may be arranged on or near the first magnet 46. The seal 68 extends circumferentially about the hole 63. In other examples, the seal 68 may be arranged on the second magnet 48. In other examples, the seal 68 can be arranged between the protrusion 62 and the cavity 66, and does not necessarily touch the magnets 46, 48. The seal 68 prevents fluid from escaping the cannula 26 at the magnetic coupling 44.

As shown in FIG. 4, the mounting piece 64 is configured to attach the second magnet 48 to the POC 22. In this example, the mounting piece 64 has two threaded holes 70 extending through two radial protrusions 72 for attachment to the POC 22. Although two holes 70 are illustrated, more or fewer may be utilized. Although the holes 70 are threaded, other attachment mechanisms may be used to secure the mounting piece 64 to the POC 22.

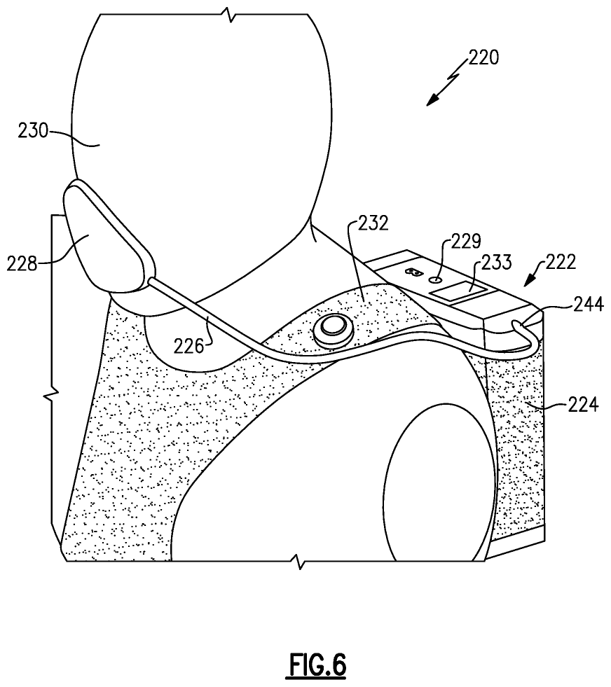

FIG. 5 illustrates another example oxygen system 120, and includes a breakout schematically illustrating the detail of another magnetic coupling. In this example, the magnetic coupling 144 is arranged along the cannula 126 between the face piece 128 and the POC 122, and is not provided at the end of the cannula 126. In this example, the cannula 126 is split into a first portion 156 and a second portion 158. The first magnet 146 is secured to the first portion 156 and the second magnet 148 is secured to the second portion 158. The first and second magnets 146, 148 secure the portions 156, 158 together such that fluid can flow from the POC 122 to the face piece 128. The first and second magnets 146, 148 may be secured to the cannula 126 via an adhesive or a friction fit, for example. In some examples, the first and second magnets 146 may be secured to the cannula 126 via a coupling piece (shown in FIG. 2). In this example, the cannula 126 may be secured to the POC 122 using known methods, such as a threaded attachment or friction fit.

FIG. 6 illustrates another example oxygen system 220. A POC 222 is arranged within a case 224. In the illustrated embodiment, the case 224 is a backpack with shoulder straps 232. Buttons 229 and indicators 233 may be located on the case 224 to allow the user 230 to control the POC 222. The POC system 220 is lightweight and portable, making it ideal for recreational use. For example, the POC system 220 may be used for skiing or biking at high altitudes, where a user may need supplemental oxygen to make breathing at the high altitude easier. The system 220 may be used for other sporting activities at high altitude and extreme sporting generally. In some examples, the system 220 may be used on the sidelines during sporting events for tired athletes to recover more quickly. To this end, the system 220 may be configured to deliver oxygen purity of less than about 86% in normal conditions, making it such that the system 220 is not a medical device, and thus can be used by recreational users without a prescription. In a further embodiment, the system 220 may be configured to deliver oxygen purity of less than 85% in normal conditions.

The above-discussed cannula and magnetic coupling may be beneficial in this system. For instance, the cannula 226 quickly detaches from the POC 222 via a magnetic coupling 244. When a user 230 is engaged in recreational activities, the cannula 226 may snag on an object, such as another person, a nearby tree, or sporting equipment, as examples. Depending on the activity, such a snag could be dangerous. For example, if the user 230 is mountain biking down a hill along rough terrain and the cannula 226 snags, the user 230 may fall off the bike and sustain a serious injury. Instead, with this disclosure, such a snag would cause the magnetic coupling 244 to quickly detach the cannula 226 from the POC 222 to prevent discomfort or injury. While there may be added benefits in recreational contexts, the magnetic coupling 44, 144 provides similar benefits in everyday environments.

It should be understood that terms such as “generally,” “substantially,” and “about” are not intended to be boundaryless terms, and should be interpreted consistent with the way one skilled in the art would interpret those terms.

Although the different examples have the specific components shown in the illustrations, embodiments of this disclosure are not limited to those particular combinations. It is possible to use some of the components or features from one of the examples in combination with features or components from another one of the examples.

One of ordinary skill in this art would understand that the above-described embodiments are exemplary and non-limiting. That is, modifications of this disclosure would come within the scope of the claims. Accordingly, the following claims should be studied to determine their true scope and content.

1. A nasal cannula for an oxygen delivery system, comprising:
a cannula having an end configured to attach to an oxygen source and a face piece configured to deliver a fluid from the oxygen source to a user; and
a magnetic coupling arranged along the cannula.

2. The nasal cannula of claim 1, wherein the magnetic coupling comprises a first magnet and a second magnet.

3. The nasal cannula of claim 2, wherein the first and second magnets have a ring shape.

4. The nasal cannula of claim 3, wherein a protrusion is arranged in a center of the first magnet, and a cavity is arranged in a second center of the second magnet, the protrusion configured to engage with the cavity.

5. The nasal cannula of claim 3, wherein a seal extends about at least one of the first and second magnets.

6. The nasal cannula of claim 2, wherein the first magnet is arranged at the end of the cannula and is configured to engage with the second magnet arranged on the oxygen source.

7. The nasal cannula of claim 2, wherein the first and second magnets are arranged along the cannula between the end and the face piece.

8. The nasal cannula of claim 1, wherein the fluid is configured to travel through the magnetic coupling.

9. The nasal cannula of claim 1, wherein the magnetic coupling comprises a mounting piece having at least one hole configured to mount the magnetic coupling to the oxygen source.

10. The nasal cannula of claim 9, wherein the at least one hole is a threaded hole.

11. The nasal cannula of claim 1, wherein the magnetic coupling comprises an alignment guide.

12. The nasal cannula of claim 1, wherein the oxygen source is a portable oxygen concentrator.

13. The nasal cannula of claim 1, wherein the user is an athlete.

14. A portable oxygen concentrator system, comprising:
a portable oxygen concentrator having a filter arranged within a housing, the filter configured to draw in ambient air, remove other gases, and deliver concentrated oxygen through an outlet on the housing;
a first magnet at the outlet; and
a cannula having an end with a second magnet configured to attach to the first magnet.

15. The portable oxygen concentrator system of claim 14, wherein the cannula has a face piece at a second end opposite the end, the face piece configured to deliver the concentrated oxygen to a user.

16. The portable oxygen concentrator system of claim 14, wherein the first and second magnets have a ring shape.

17. The portable oxygen concentrator system of claim 14, wherein the first magnet extends about the outlet.

18. The portable oxygen concentrator system of claim 14, wherein a seal is arranged between the first and second magnets.

19. The portable oxygen concentrator system of claim 14, wherein the cannula is configured to deliver the concentrated oxygen to a user, and the user is an athlete.

20. The portable oxygen concentrator system of claim 14, wherein the portable oxygen concentrator is configured to deliver the concentrated oxygen at a purity of less than 86%.
